(19) United States
(12) Patent Application Publication
(10) Pub. No.: US 2016/0066973 A1
(43) Pub. Date: Mar. 10, 2016

(54) COLLAPSIBLE TELESCOPING SLAPHAMMER

(71) Applicant: Zimmer, Inc., Warsaw, IN (US)

(72) Inventors: Paul Borries, Columbia City, IN (US); Aaron Robert Gorsuch, Warsaw, IN (US)

(21) Appl. No.: 14/822,502

(22) Filed: Aug. 10, 2015

Related U.S. Application Data
(60) Provisional application No. 62/048,399, filed on Sep. 10, 2014.

(51) Int. Cl. A61B 17/92

(52) U.S. Cl. CPC A61B 17/92; A61B 2017/922; A61B 2017/00991

(57) ABSTRACT

A collapsible slap hammer tool is disclosed herein for use in the insertion, removal or other manipulation of an object coupled thereto. The collapsible slap hammer tool comprises a guide rod engaged with a telescoping handle assembly including two or more telescoping members. The guide rod can be telescopically retracted within or extended from the telescoping handle assembly. The guide rod can mate with an object such that in response to a user manipulation the telescoping handle assembly generates a force on the guide rod and a corresponding force on an object coupled to the guide rod, for insertion, removal or other manipulation of the object coupled thereto.

### CLAIM OF PRIORITY

This application claims the benefit of U.S. Provisional Patent Application Ser. No. 62/048,399, filed on Sep. 10, 2014, the benefit of priority of which is claimed hereby, and which is incorporated by reference herein in its entirety.

### BACKGROUND

In orthopedic surgical procedures various tools and instruments are used to remove damaged or diseased bone, prepare the bone for the insertion of prosthetic implant components, to remove instruments that have been temporarily affixed to the bone or to remove previously placed or temporary prosthetic implant components. Certain tools used during these procedures provide a force toward or force away from the surgical area to assist in the surgical procedure. For example, “slap hammers” (also known as “slaphammers” or “slide hammers”) have been developed and are widely used in orthopedic procedures to apply an impacting force on various tools used during surgery. Slap hammers typically consist of a guide rod and a sliding weight. One end of the guide rod is affixed to an object, such as a device or a surgical instrument. The sliding weight may be thrown upward, away from the surgical site, generating a force when the sliding weight strikes a stop on the end of the guide rod. The sliding weight may be repeatedly “thrown” away from the surgical site to extract the device or the instrument attached to the guide rod. Alternatively, the hammer may be dropped or “thrown” downward, toward the surgical site to impact a stop at end of the guide rod that is attached to the device or instrument.

A slap hammer tool for an orthopedic procedure includes U.S. Pat. No. 6,814,738, which discloses a handle portion and a substantially cylindrical, hollow slideable mass disposed around the handle portion and mated to a surgical tool such that when the mass is positioned in a first orientation it may be used to generate a force, and when the mass is positioned in a second orientation the mass may be removed from the slap hammer tool. U.S. Pat. No. 6,709,439 discloses a hollow tube, a handle slideably disposed around the tube and a mass coupled to the handle and disposed within the hollow tube such that movement of the handle causes simultaneous movement of the mass to deliver a force. U.S. Pat. No. 8,486,084 discloses a guide rod defining a longitudinal axis, a striking member disposed on the guide rod and selectively positionable at various locations along the guide rod, and a sliding weight slideable along the guide rod between a first end of the guide rod and the striking member.

Current slap hammers used in orthopedic surgical procedures, for example to remove a femoral stem implant in hip replacement surgery, require a tool that produces significant momentum along an axis of the femoral stem implant to break the implant fixation to the bone. Thus, slap hammer tools used in orthopedic surgical procedures are large and heavy in order to generate sufficient momentum and force, making the devices difficult to use and requiring significant space for storage and transport. Accordingly, there remains a need for an improved slap hammer device that is compact for ease of handling and storage, yet provides enough momentum or “throw” to effectively apply an insertion force or removal force to an object, such as a surgical device or a surgical instrument.

### OVERVIEW

A slap hammer tool includes a guide rod, including a first end configured for coupling to an object, and a second end coupled to a handle assembly. The handle assembly includes a first member and a second member slideably movable relative to each other. In certain embodiments, the handle assembly can also be slideably movable relative to the guide rod. In some embodiments one or both of the first member and the second member of the handle assembly can comprise a weight or be formed of a weighted or heavy material.

The handle assembly can be actuated by a user by sliding the handle assembly along an axis of the guide rod to generate momentum and to impact a surface or feature of the guide rod, or apply a force through the guide rod to a device or instrument coupled to the guide rod, such as a surgical instrument or a surgical device. In certain embodiments the handle assembly can include a drive feature to assist coupling and uncoupling of the guide rod with an object, such as a surgical instrument or a surgical device.

In certain embodiments a slap hammer tool system includes a guide rod, configured for coupling to an object, and a telescoping handle assembly couplable to the guide rod. The telescoping handle assembly includes at least two members telescopically engaged with one another such that one member is receivable or retractable within, and extendable from, the other member. The telescoping handle assembly allows the handle members to be stored, one within the other, providing a compact handle for use with the guide rod. In certain embodiments the telescoping handle assembly can include a drive feature to assist coupling and uncoupling of the guide rod with an object, such as a surgical instrument or a surgical device. One or more of the telescoping segments of the handle assembly can comprise a weight or be formed of a heavy material.

In other embodiments a slap hammer tool system includes a guide rod configured for coupling to an object and configured for telescopic engagement with a telescoping handle assembly, such that the guide rod is receivable or retractable within the handle assembly and extendable from the handle assembly. The guide rod when telescopically coupled to the telescoping handle assembly allows at least a portion of the guide rod to be stored within the handle assembly, providing a compact slap hammer tool for storage and transport.

The telescoping movement of the handle assembly in relation to the guide rod can generate an impacting force when one or more of the telescoping handle members are slideably moved in relation to each other and in relation to the guide rod. Extension of the telescoping handle members away from the guide rod extends the distance the handle assembly can be moved away from guide rod, creating greater momentum and allowing a longer “throw” or force to extract an object, such as an instrument or device coupled to the guide rod of the slap hammer.

The telescoping segments of the handle assembly can nest, one within the other, in a collapsed configuration. The telescoping handle assembly telescopically coupled to the guide rod permits at least a portion of the guide rod to nest within the handle assembly, providing a compact slap hammer tool for storage and transport.

In Embodiment 1, a slap hammer tool system can comprise a guide rod and a handle assembly configured to be coupled to the guide rod. The handle assembly can include a first member and a second member, the first member telescopically movable relative to the second member.

In Embodiment 2, the slap hammer tool system of Embodiment 1 can be optionally configured such that the handle assembly is configured to be telescopically coupled to the guide rod.

In Embodiment 3, the slap hammer tool system of any one or each of Embodiments 1 and 2, can be optionally configured such that in a coupled configuration the guide rod is telescopically movable relative to one or both of the first member and the second member.

In Embodiment 4, the slap hammer tool system of any one or more of Embodiments 1 through 3 can be optionally configured such that in coupled configuration at least a portion of the guide rod is nested within a chamber of the first member and at least a portion of the first member is nested within a chamber of the second member.

In Embodiment 5, the slap hammer tool system of any one or more of Embodiments 1 through 4 can be optionally configured such that in coupled configuration at least a portion of the guide rod is telescopically extended from a chamber of the first member and at least a portion of the first member is telescopically extended from a chamber of the second member.

In Embodiment 6, the slap hammer tool system of any one or more of Embodiments 1 through 5 can be optionally configured such that in a coupled configuration the first member is configured to impact a striking feature of the guide rod when the first member is telescopically moved relative to the guide rod.

In Embodiment 7, the slap hammer tool system of any one or more of Embodiments 1 through 6 can be optionally configured such that the second member is configured to impact a striking feature of the first member when the second member is telescopically moved relative to the first member.

In Embodiment 8, the slap hammer tool system of any one or more of Embodiments 1 through 7 can be optionally configured such that the first member includes a first stop and a second stop, and the second member is configured to impact the first stop when the second member is telescopically moved in a first direction relative to the first member, and the second member is configured to impact the second stop when the second member is telescopically moved in a second direction, opposite the first direction, relative to the first member.

In Embodiment 9, the slap hammer tool system of any one or more of Embodiments 1 through 8 can be optionally configured such that the first member comprises a first segment and a second segment, the first segment is configured to be telescopically movable relative to the second segment, and the second segment is configured to be telescopically movable relative to the second member.

In Embodiment 10, a slap hammer tool can comprise a guide rod and a handle assembly coupled to the guide rod. The handle assembly can include a first member and a second member slideably movable relative to each other in response to a user manipulation.

In Embodiment 11, the slap hammer tool of Embodiment 10 can be optionally configured such that the first member is at least partially received within a chamber of the second member.

In Embodiment 12, the slap hammer tool of any one or each of Embodiments 10 and 11 can be optionally configured such that the guide rod is at least partially received within a chamber of the handle assembly.

In Embodiment 13, the slap hammer tool of any one or more of Embodiments 10 through 12 can be optionally configured such that the first member, the second member, or both, is slideably movable relative to the guide rod in response to the user manipulation.

In Embodiment 14, the slap hammer tool of any one or more of Embodiments 10 through 13 can be optionally configured such that the handle assembly is slideably moveable in a first direction to a first stop coupled to the guide rod, and the handle assembly is slideably moveable in a second direction, opposite the first direction, to a second stop coupled to the guide rod.

In Embodiment 15, the slap hammer tool of any one or more of Embodiments 10 through 14 can be optionally configured such that the guide rod includes a striking feature, the handle assembly further includes an impacting feature configured to contact the striking feature when the handle assembly is slideably moved relative to the guide rod.

In Embodiment 16, the slap hammer tool of any one or more of Embodiments 10 through 15 can be optionally configured such that an impacting feature is disposed adjacent an interior chamber of the handle assembly.

In Embodiment 17, the slap hammer tool of any one or more of Embodiments 10 through 15 can be optionally configured such that an impacting feature is disposed on an exterior surface of the handle assembly.

In Embodiment 18, a method can comprise coupling a slap hammer tool to an object, the slap hammer including a guide rod and a telescoping handle assembly coupled to the guide rod; and slideably moving the handle assembly in a direction to apply a force to the object in the direction of slideable movement of the handle assembly. The method can further include coupling a telescoping handle assembly to a guide rod, the telescoping handle assembly can include a first member and a second member telescopically engaged with each other.

In Embodiment 19, the method of Embodiment 18 can be optionally modified such that the step of slideably moving includes slideably moving the first member or the second member relative to the guide rod.

In Embodiment 20, the method of any one or each of Embodiments 18 and 19 can be optionally modified such that slideably moving the first member or the second member relative to the guide rod includes slideably moving the first member or the second member away from the object or toward the object.

In Embodiment 21, the device and methods of any one (or portion of any one) or any combination of Embodiments 1-20 is optionally configured such that all elements or options recited are available to use or select from.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a isometric image of a slap hammer tool in a fully extended configuration, according to an embodiment;

FIGS. 2A, 2B illustrate a cross-sectional view of a slap hammer tool in a fully extended configuration, according to an embodiment;

FIG. 3 illustrates an isometric image of a slap hammer tool in a collapsed configuration, according to an embodiment;

FIG. 4 illustrates a cross-sectional view of a slap hammer tool in a collapsed configuration, according to an embodiment;

FIG. 5 illustrates a cross-sectional view of a slap hammer tool in a collapsed configuration, according to an embodiment.

### DETAILED DESCRIPTION

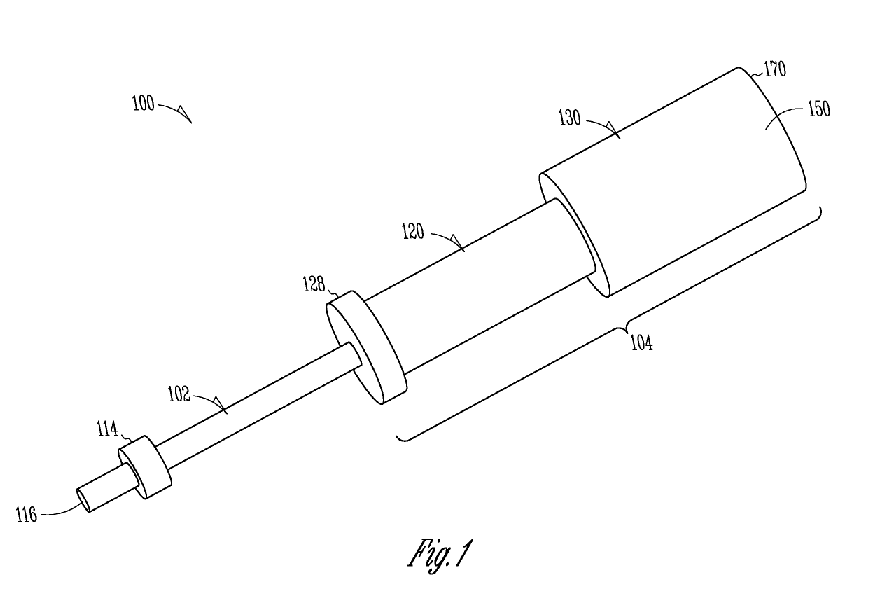

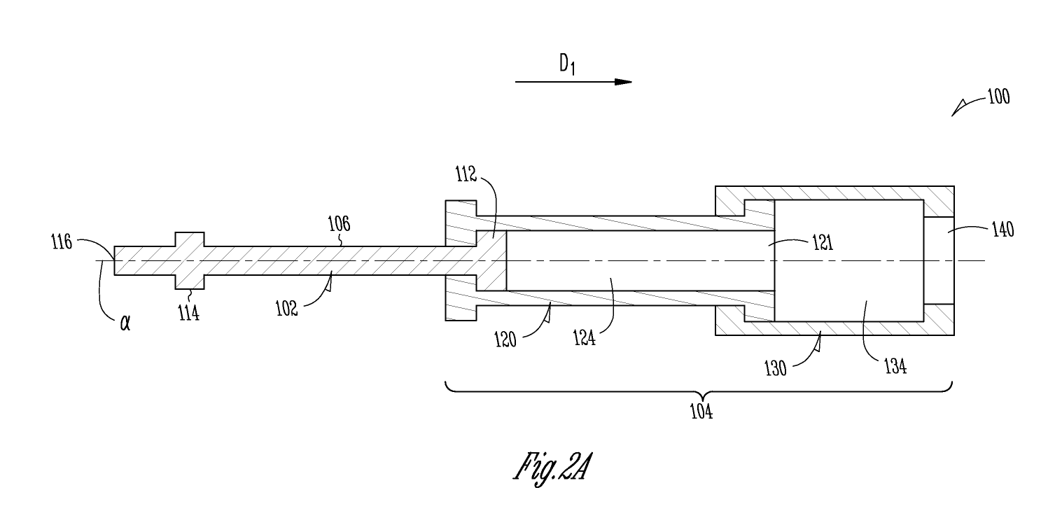

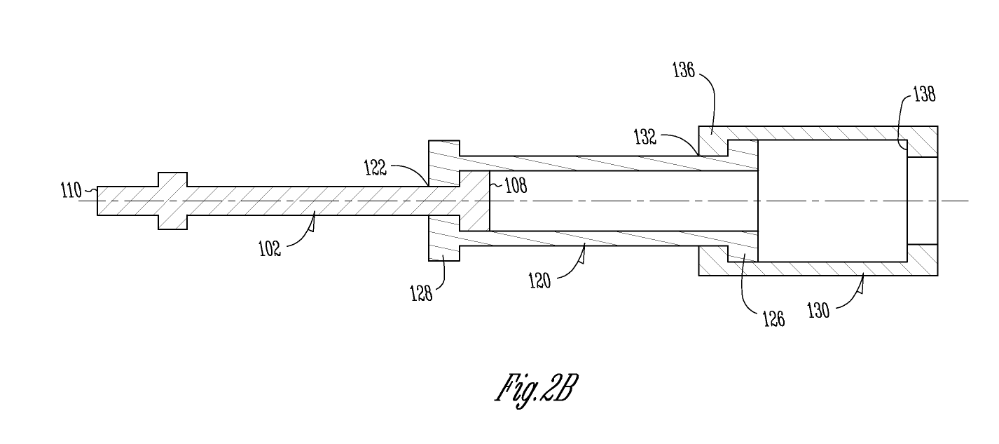

Configurations and methods of use for a slap hammer tool are disclosed herein. In one embodiment, a slap hammer is configured for engagement and disengagement with an object, such as an instrument or device used in connection with a surgical procedure. For example, an engagement of the slap hammer tool with an object, such as a surgical instrument or a surgical device, can be used to insert, place, extract, reposition, or otherwise manipulate the object. FIGS. 1, 2A, 2B, 3, 4 and 5 illustrate a slap hammer tool 100 configured for releasable engagement with an object according to one or more embodiments. As best viewed in FIGS. 1, 2A and 2B, the slap hammer tool 100 can include a connecting portion 102 and a handle assembly 104. As further described herein, the slap hammer tool 100 can include an engagement member or engagement feature 116 located at a connecting end 110 of the slap hammer tool 100. The engagement member 116 can be configured for detachable coupling of the slap hammer tool 100 to an object, such as an instrument or device, thereby allowing the slap hammer tool 100 to apply force to the object coupled thereto. In one or more embodiments of a slap hammer tool 100 described in more detail below a handle assembly 104 can include a gripping member or gripping surface or gripping feature 150 to facilitate holding the slap hammer tool 100 or actuating the handle assembly 104.

A connecting portion 102 of a slap hammer tool 100 can extend between a first end 108 and a second end 110 and can include a shaft or a guide rod 106 having a length extending between the first end 108 and the second end 110 of the connecting portion 102. A guide rod 106 of the connecting portion 102 can include a hollow body or a solid body.

In one ore more embodiments a connecting portion 102 can include a stop 112, 114 disposed on or coupled to the connection portion 102. In this configuration, an impacting feature 128, 136, 138 of the handle assembly 104 can be configured to contact a stop 112, 114 disposed on or coupled to the connecting portion 102. In some embodiments a stop 112 can be located at or adjacent to a first end 108 of the connecting portion 102. In some embodiments a stop 114 can be located at or adjacent to a second end 110 of the connecting portion 102. In other embodiments a slap hammer tool 100 can include both a first stop 112 and a second stop 114. In certain embodiments a first stop can be located at or adjacent to a first end 108 of the connecting portion 102 and a second stop 114 can be located at or adjacent to a second end 110 of the connecting portion 102.

In one or more embodiments a slap hammer tool 100 can include a stop 112, 126 located within an interior space or an interior chamber 124, 134 of a handle assembly 104. In this configuration, an impacting feature 128, 136,138 of the handle assembly 104 located adjacent an interior chamber 124, 134 can be configured to contact a stop 112, 126 that is located within an interior chamber 124, 134 of the handle assembly 104.

In one or more embodiments a handle assembly 104 of a slap hammer tool 100 can include a slideable first member 120 and a slideable second member 130. In some embodiments one or both of the slideable first member 120 and slideable second member 130 can be configured for slideable movement relative to the connecting portion 102. In some embodiments the slideable first member 120 and the slideable second member 130 can be configured to be independently slideable relative to the connecting portion 102. In some embodiments the slideable first member 120 and the slideable second member 130 can be configured to be slideable relative to each other. In some embodiments one or both of a slideable first member 120 and a slideable second member 130 of a handle assembly 104 can include a weight or be fabricated from a weight or a heavy material.

Therefore, as a handle assembly 104 is slideably actuated by a user relative to a connecting portion 102, such as by sliding one or both of a slideable first member 120 and a slideable second member 130 of the handle assembly 104 in a direction along an axis a of the connecting portion 102, an impacting feature 128, 136, 138 of the handle assembly 104 can contact a stop 112, 126 disposed on or coupled to the connecting portion 102 or the first member 120 of the handle assembly 120. When the impacting feature 128, 136, 138 of the handle assembly 104 contacts a stop 112, 126 a force f in the direction of the slideable movement of the slideable member 120, 130 can be applied to the stop 112, 126, the force f transmitted to a second end 110 of the connecting portion 102 and an object engaged with the connecting portion 102, for example an object coupled to the connecting portion 102 through a connecting feature 116 of the slap hammer tool 100.

In some embodiments a handle assembly can be actuated by slideable movement of a slideable member 120, 130 of the handle assembly 104 in a first direction D1 along an axis α of the connecting portion 102 to apply a force f to a stop 112, 126 disposed on or coupled to the connecting portion 102 or the first member 120. In other embodiments a handle assembly 104 can be actuated by slideable movement of a slideable member 120, 130 of the handle assembly 104 in a second direction D2, opposite the first direction D1, along an axis α of the connecting portion 102 to apply a force f to a stop 114 disposed on or coupled to the connecting portion 102. It will be understood that in any embodiment a handle assembly 104 and a connecting portion 102 can be configured such that slideable movement of a slideable member 120, 130 of the handle assembly 104 in relation to the connecting portion 102 can include slideable movement in one or both of the first direction D1 and the second direction D2.

As illustrated in FIGS. 1, 2A, 2B, 3 and 4, in one or more embodiments a slap hammer tool 100 can include a connecting portion 102, as previously described, coupled to a telescoping handle assembly 104. In one or more embodiments a telescoping handle assembly 104 can include a first member 120 and a second member 130 configured to be telescopically coupled with the first member 120. In one or more embodiments of a telescoping handle assembly 104 a first member 120 of the handle assembly 104 can be configured to nest at least partially within a second member 130 of the handle assembly 104. A telescoping handle assembly 104 can have a first configuration in which a first member 120 is telescopically received or retracted at least partially within a second member 130, and a second configuration in which the first member 120 is telescopically extended at least partially from the second member 120.

Thus, a telescoping first member 120 and a telescoping second member 130 of a telescoping handle assembly 104 can be configured to be telescopically slideable relative to each other. In some embodiments one or both of the first member 120 and second member 130 can be configured for slideable movement relative to the connecting portion 102. In some embodiments the first member 120 and the second member 130 can be configured to be independently slideable relative to the connecting portion 102. In some embodiments one or both of a slideable first member 120 and slideable second member 130 of a telescoping handle assembly 104 can include a weight or be fabricated from a weight or a heavy material.

In some embodiments a connecting portion 102 of a slap hammer tool 100, as previously described, can be telescopically engaged or coupled with a telescoping handle assembly 104. In some embodiments, a connecting portion 102 can be configured to be telescopically engaged with a telescoping handle assembly 104. In some embodiments a connecting portion 102 can be configured to be received or retracted at least partially within one or both of a first member 120 and a second member 130 of a telescoping handle assembly 104. In some embodiments a connecting portion 102 can be configured to at least partially nest within one or both of the first member 120 and the second member 130 of a telescoping handle assembly 104.

In some embodiments a slap hammer tool 100 can include a connecting portion 102, as previously described, configured to be received at least partially within a first member 120 of a handle assembly 104 and the first member 120 of the handle assembly 104 can be configured to be received at least partially within a second member 130 of the handle assembly 104. In some embodiments a connecting portion 102 can be configured to nest at least partially within a first member 120 of a telescoping handle assembly 104 and the first member 120 can be configured to nest at least partially within a second member 130 of a telescoping handle assembly 104. In still other embodiments, a guide rod 106 of connecting portion 102 can be configured to nest within a first member 120 of a telescoping handle assembly 104 and the first member 120 can be configured to be nested at least partially within a second member 130 of a telescoping handle assembly 104.

A telescoping handle assembly 104 can include a bore 122, 132 and an interior chamber 124, 134. A bore 122, 132 and a interior chamber 124, 134 of the telescoping handle assembly 104 can be sized and dimensioned to receive at least a part of a connecting portion 102 and can be configured for coupling the handle assembly 104 to the connecting portion 102.

In one or more embodiments a telescoping handle assembly 104 can be disposed over a first end 108 of a connecting portion 102 and the first end 108 of the connecting portion 102 can be configured to be received through the bore 122, 132 such that the handle assembly 104 surrounds at least the first end 108 of the connecting portion 102. In other embodiments a first end 108 of a connecting portion 102 can be configured to pass through a bore 122, 132 and be received within an interior chamber 124, 134 of a handle assembly 104. In some embodiments the telescoping handle assembly 104 can be configured to surround at least a portion of the guide rod 106 such that at least a portion of the guide rod 106 is received within a bore 122, 132 and is surrounded by an interior chamber 124, 134 of the telescoping handle assembly 104. In some embodiments a first end 108 of a connecting portion 102 and at least part of a guide rod 106 of a connecting portion 102 can be configured to nest at least partially within an interior chamber 124, 134 of the telescoping handle assembly 104.

A telescoping handle assembly 104 of a slap hammer tool 100 can include one or more substantially hollow or tubular telescoping members 120, 130. In one example shown in FIGS. 1, 2A, 2B, 3 and 4, a telescoping handle assembly includes two telescoping members 120, 130. A telescoping first member 120 can be configured to fit, in a sliding fashion, within a telescoping second member 130. In some embodiments a first member 120 of a telescoping handle assembly 104 can have a smaller diameter than a second member 130 of a telescoping handle assembly 104, which can allow the first member 120 to be telescopically slideable within the second member 130 of the telescoping handle assembly 104.

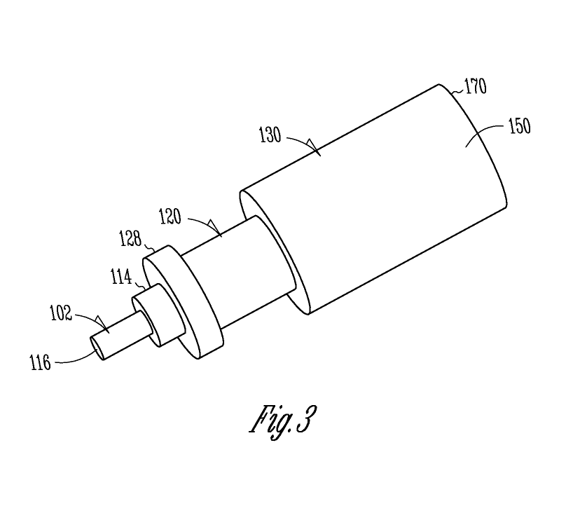

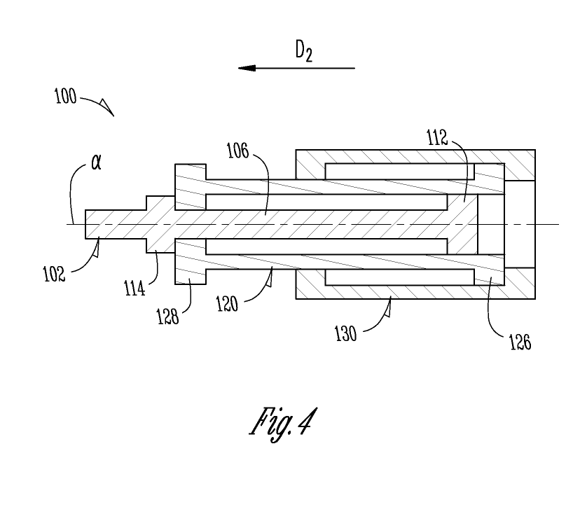

FIGS. 1, 2A and 2B illustrate an embodiment of a telescoping slap hammer tool 100 including a telescoping handle assembly 104 in an extended configuration. FIGS. 3 and 4 illustrate an embodiment of a telescoping slap hammer tool 100 including a telescoping handle assembly 104 in a collapsed or retracted configuration.

In some embodiments a telescoping handle assembly 104 can include at least one tubular telescopic member 120, 130. In other embodiments a telescoping handle assembly 104 can include at least two tubular members 120, 130. A first member 120 of a telescoping handle assembly 104 can be configured to be received within a tubular second member 130 of a telescoping handle assembly 104. In other embodiments, a telescoping handle assembly 104 can include at least two tubular members 120, 130, such that a tubular first member 120 can be configured to be slideably received within the tubular second member 130. In this configuration, a connecting portion 102 can be configured to be slideably received within one or both of the tubular first member 120 and tubular second member 130 of the telescoping handle assembly 104 to provide a telescoping slap hammer tool 100.

It will be appreciated that one or both of a first member 120 and a second member 130 of a telescoping handle assembly 104, as described above, can include two or more telescopic segments, for example first member 120 can include telescopic segments 120A, 120B that can be configured to slideably fit one within another, and second member 130 can include telescopic segments 130A, 130B that can be configured to slideably fit one within another, and in turn the largest first member telescopic segment 120A, 120B can be configured to be slideably received at least partially within the smallest second member telescopic segment 130A, 130B.

In some embodiments a first telescopic segment 120A, 130A can have a smaller diameter than a second telescoping segment 120B 130B, which may allow a first telescopic segment 120A, 130A to be telescopically slideable within the second telescopic segment 120B, 130B. In an example, telescopic segment 120A has a smaller diameter than telescopic segment 120B and telescopic segment 120A can be slideably received within telescopic segment 120B, while telescopic segment 120B can have a smaller diameter than second member 130 or second member telescopic segment 130A, 130B, and first member telescopic segment 120B can be slideably received within second member 130 or second member telescopic segment 130A, 130B. Similarly, second member first telescopic segment 130A can have a smaller diameter than second member second telescopic segment 130B and can be slideably received within second member second telescopic segment 130B.

It will be appreciated that more or fewer telescopic segments 120A, 120B of a first member 120 and more or fewer telescopic segments 130A, 130B of a second member 130 of a telescoping handle assembly 104 can be provided as required or desirable for a particular use. In particular, the number of telescopic segments of a first member 120 and a second member 130 can be varied as required or desired to provide the desired “throw”, momentum or force to manipulate an object coupled to the slap hammer tool 100. Also, the length of any telescopic segment of a first member 120 and a second member 130 of a telescoping handle assembly 104 can be varied as required or desired to provide the desired “throw”, momentum or force to manipulate an object coupled to the slap hammer tool 100.

As shown in FIGS. 1, 2A and 2B, a first member 120 and a second member 130 of a telescoping handle assembly 104 can be configured as cylindrical elongate tubes. In some embodiments a first member 120 and a second member 130 of a telescoping handle assembly 104 can be configured in any shape of an elongate tube, such as square, rectangular, oval, elliptical, or triangular elongate tubes. A first member 120 and a second member 130 of a telescoping handle assembly 104 can be formed of any type or any combination of materials, such as a metal, an alloy, a plastic, wood, or any other suitable material to provide a required or desired mass to provide the desired momentum and force to manipulate an object coupled to a slap hammer tool 100. A first member 120 and a second member 130 of a telescoping handle assembly 104 can be of any length, and a first member 120 of a telescoping handle assembly 104 can be have a different length than a second member 130 of a telescoping handle assembly 104.

In one or more embodiments a telescoping slap hammer tool 100 can include a connecting portion 102 telescopically coupled to a telescoping handle assembly 104 as described above. In an embodiment at least a first end 108 of a connecting portion 102 can be configured to be slideably received at least partially within a tubular first member 120 of a handle assembly 104 and the first member 120 of the handle assembly 104 can be configured to be slideably received at least partially within a tubular second member 130 of the handle assembly 104. The telescoping slap hammer tool 100 can be slideably moved between a collapsed or retracted configuration, for example as shown in FIGS. 3 and 4, and extended configuration, for example as shown FIGS. 1, 2A and 2B.

In some embodiments a connecting portion 102 of a telescoping slap hammer tool 100 can include a stop 114 configured to prevent connecting portion 102 from being completely received or retracted within telescoping handle assembly 104. In some embodiments a connecting portion 102 can include a stop 112, 126 configured to prevent connecting portion 102 and a first member 120 from being completely extended from an interior chamber 124, 134 of a telescoping handle assembly 104. A stop 112, 114 can be configured to prevent the connecting portion 102 from being completely received or retracted within an interior chamber 124, 134 of one or both of a first member 120 or a second member 130 of a telescoping handle assembly 104. A stop 112 can be configured to prevent the connecting portion 102 from being completely extended from an interior chamber 124, 134 of one or both of a first member 120 or a second member 130 of a telescoping handle assembly 104. A stop 126 can be configured to prevent the first member 120 from being completely extended from an interior chamber 134 of a second member 130 of a telescoping handle assembly 104. A stop 128 can be configured to prevent the first member 120 from being completely received within the second member 130 of a telescoping handle assembly 104. In some embodiments an interior chamber 124, 134 of a telescoping handle assembly 104 can include one or more impacting surfaces or features 128, 136, 138 configured to contact a stop 112, 126 that is received within an interior chamber 124, 134 of the telescoping handle assembly 104.

In some embodiments a first member 120 of a telescoping handle assembly 104 can include a first stop 126 and a second stop 128. A first member first stop 126 can be configured to prevent a first member 120 of a handle assembly 104 from being completely extended from an interior chamber 134 of a second member 130 of a handle assembly 104. A first member second stop 128 can be configured to prevent a first member 120 from being completed received or retracted within an interior chamber 134 of a second member 130 of a handle assembly 104. A first member first stop 126 can also be configured to contact an impacting feature 136 disposed adjacent to an interior chamber 134 of a second member 130 of the handle assembly 104. A first member 120 can also include a first member second stop 128 configured to contact or impact a stop 112, 114 disposed on or coupled to a connecting portion 102.

Therefore, as a telescoping handle assembly 104 is slideably actuated by a user relative to a telescoping connecting portion 102 of a slap hammer tool 100, such as by sliding one or both of a first member 120 and a second member 130 of the telescoping handle assembly l04, concurrently or consecutively, in a direction along an axis α of the connecting portion 102 an impacting feature 128, 136, 138 of the handle assembly 104 can contact a stop 112, 114, 126 disposed on or coupled to the connecting portion 102 or a first member 120. When an impacting feature 128, 136, 138 of the handle assembly 104 contacts a stop 112, 114, 126 a force f in the direction of the slideable movement of a telescoping member 120, 130 of the handle assembly 104 can be applied to the stop 112, 114, 126, the force f being transmitted to a second end 110 of the connecting portion 102 and an object engaged with or coupled to the connecting portion 102, for example an object coupled to the connecting portion 102 through a engagement or connecting feature 116 of the slap hammer tool 100.

In some embodiments a telescoping handle assembly 104 can be actuated by slideable movement of a telescoping member 120, 130 of the handle assembly 104 in a first direction D1 along an axis α of the connecting portion 102 such that an impacting feature 128, 136, 138 disposed adjacent to an interior chamber 124, 134 of handle assembly 104 can apply a force f to a stop 112, 114, 126 disposed on or coupled to the connecting portion 102 or a first member 120. In some configurations a stop 112, 126 can be positioned within an interior chamber 124, 134 of the handle assembly 104.

In other embodiments a telescoping handle assembly 104 can be actuated by slideable movement of a slideable member 120, 130 of the handle assembly 104 in a second direction D2, opposite the first direction D1, along an axis α of the connecting portion 102, such that an impacting feature 128 disposed on an exterior surface of the handle assembly 104 can apply a force f to a stop 112, 114, disposed on or coupled to the connecting portion 102, and such that an impacting feature 136 of the second member 130 of the handle assembly 104 can apply a force f to a stop 128, disposed on or coupled to the first member 120. It will be understood that in any embodiment a handle assembly 104 and a connecting portion 102 can be configured such that slideable movement of a slideable member 120, 130 of the handle assembly 104 in relation to the connecting portion 102 can include slideable movement in one or both of the first direction D1 and the second direction D2.

In one or more embodiments a slap hammer tool 100 can include a connecting portion 102, as previously described, coupled to a handle assembly 104 that is configured for slideable movement relative to the connecting portion 102. In some embodiments a first member 120 and a second member 130 of the handle assembly 104 can each be configured for slideable movement relative to the connecting portion 102. In some embodiments a slideable handle assembly can include a first member 120 and a second member 130 that can be configured for slideable movement relative to each other. In some embodiments a first member 120 can be configured for slideable movement relative to a connecting portion 102 independently of slideable movement of a second member 130 relative to the connecting portion 102. In other embodiments a slideable second member 130 can be configured for slideable movement relative to a connecting portion 102 independently of slideable movement of a first member 120 relative to the connecting portion 102. One or both of a slideable first member 120 and a slideable second member 130 of the handle assembly 104 can include tubular segments. In some embodiments a slideable handle assembly 104, including one or both of the slideable first member 120 and the slideable second member 130, can include a weight or be fabricated from a weight or a heavy material.

In one or more embodiments a slideable handle assembly 104 can be configured to be disposed about a connecting portion 102. In some embodiments a handle assembly 104 can be configured to be disposed about a first end 108 of a connecting portion. In some embodiments a handle assembly 104 can be configured to surround at least a portion of a guide rod 106 of a connecting portion 102. In some embodiments a first member 120 of a slideable handle assembly 104 can be configured to receive a first end 108 of a connecting portion 102. In some embodiments a first member 120 of a slideable handle assembly 104 can be configured to receive at least a portion of a guide rod 106 of a connecting portion 102.

In some embodiments a second member 130 of a slideable handle assembly 104 can be configured to be disposed about a first member 120 of the slideable handle assembly. In some embodiments a second member 130 of a handle assembly 104 can be configured to be disposed about a first end 121 of a first member 120 of a slideable handle assembly. In other embodiment a second member 130 of a slideable handle assembly 104 can be configured to surround at least a portion of a first member 120 of the slideable handle assembly 104. In some embodiments a second member 130 of a slideable handle assembly 104 can be configured to receive at least a portion of a first member 120 of the slideable handle assembly 104.

Therefore, as a slideable handle assembly 104 is slideably actuated by a user, such as by sliding a second member 130 of the slideable handle assembly 104 over at least a portion of a first member 120 of the slideable handle assembly 104 and by sliding, concurrently or consecutively with sliding of the second member 130, a first member 120 of the handle assembly 104 along an axis α of the connecting portion 102, the first member 120 of the handle assembly 104 can contact a stop 112, 114 disposed on or coupled to the connecting portion 102. When the first member 120 of the slideable handle assembly contacts a stop 112, 114 of the connecting portion 102, a force f in the direction of the slideable movement of the first member 120 can be applied to the stop 112, 114, the force being transmitted to a second end 110 of the connecting portion 102 and an object engaged with the connecting portion 102, for example an object coupled to the connecting portion 102 through a engagement or connecting feature 116 of the slap hammer tool 100.

In some embodiments a slideable handle assembly 104 configured to be slideably coupled to a connecting portion 102 can be actuated by slideable movement of a second member 130 of a handle assembly 104 over a at least part of a first member 120 of the handle assembly 104 to contact a stop 126, 128 disposed on or coupled to the first member 120 of the slideable handle assembly 104. When the second member 130 of the handle assembly contacts a stop 126, 128 on the first member 120 of the handle assembly 104, a force f in the direction of the slideable movement of the second member 130 can be applied to the stop 126, 128, the force being transmitted to a second end 110 of the connecting portion 102 and an object engaged with the connecting portion 102, for example an object coupled to the connecting portion 102 through an engagement or connecting feature 116 of the slap hammer tool 100.

In some embodiments a slideable handle assembly 104 can be actuated by slideable movement of a second member 130 of the handle assembly 104 in a first direction D1 over at least a portion of a first member 120 of the slideable handle assembly 104 to apply a force f in the direction D1 to a stop 126, 128 disposed on or coupled to the first member 120, and slideable movement of a first member 120 of a handle assembly 104 in the first direction D1 along an axis α of the connecting portion 102 to apply a force f in the direction D1 to a stop 112, 114 disposed on or coupled to the connecting portion 102. When the second member 130 of the slideable handle assembly contacts a stop 126, 128 on the first member 120 of the slideable handle assembly 104 and when the first member 120 of the slideable handle assembly 120 contacts a stop 112, 114 on the connecting portion 102, a force f in the direction D1 can be applied, respectively, to stops 126, 128 and stop 112, 114 the force continuing in a direction toward a second end 110 of the connecting portion 102 and toward an object engaged with the connecting portion 102, for example an object coupled to the connecting portion 102 through a connecting feature 116 of the slap hammer tool 100.

In some embodiments a handle assembly 104 can be actuated by slideable movement of a second member 130 in a second direction D2, opposite the first direction D1, over a portion a first member 120 of the slideable handle assembly 104 to apply a force f in the direction D2 to a stop 126, 128 disposed on or coupled to the first member 120, and slideable movement of the first member 120 of a handle assembly 104 in the second direction D2 along an axis α of the connecting portion 102 to apply a force f in the direction D2 to a stop 112, 114 disposed on or coupled to the connecting portion 102. When the second member 130 of the handle assembly contacts a stop 128, 128 of the first member 120 and when the first member 120 of the slideable handle assembly contacts a stop 112, 114 of the connecting portion 102, a force f in the direction D2 can be applied, respectively to stop 126, 128 and stop 112, 114 the force continuing in a direction toward a second end 110 of the connecting portion 102 and toward an object engaged with the connecting portion 102, for example an object coupled to the connecting portion 102 through a connecting feature 116 of the slap hammer tool 100. It will be understood that any embodiment of a handle assembly 104 and a connecting portion 102 as described herein can be configured such that slideable movement of the handle assembly 104 in relation to the connecting portion 102, including slideable movement of a slideable first member 120 and a slideable second member 130 in relation to a connecting portion 102, can include slideable movement in one or both of a first direction D1 and a second direction D2.

In one or more embodiments of a slap hammer tool 100 a connecting portion 102, as previously described, can be configured to be detachably coupled to a handle assembly 104 configured for slideable movement relative to a connecting portion 102. The handle assembly 104 can include a first member 120 configured to detachably couple the handle assembly 100 to the connecting portion 102, and a second member 130 configured for slideable movement relative to the first member 120 and relative to the connecting portion 102 when the handle assembly is detachably coupled to the connecting portion 102. One or both of first member 120 and a second member 130 of the handle assembly 104 can include tubular segments. In some embodiments a first member 120 of the handle assembly 104 can be configured to be slideably received at least partially within a tubular second member 130 of the handle assembly 104. In other embodiments a handle assembly 104 can include at least two tubular members 120, 130 such that a first member 120 can be configured to be slideably received within the second member 130.

As shown in FIGS. 1, 2A and 2B, a first member 120 and a second member 130 of a slideable handle assembly 104 can be configured as cylindrical elongate tubes. In some embodiments a first member 120 and a second member 130 of a slideable handle assembly 104 can be configured in any shape of an elongate tube, such as square, rectangular, oval, elliptical, or triangular elongate tubes. A first member 120 and a second member 130 of a slideable handle assembly 104 can be formed of any type or any combination of materials, such as a metal, an alloy, a plastic, wood, or any other suitable material to provide a required or desired mass to provide the desired momentum and force to manipulate an object coupled to a slap hammer tool 100. A first member 120 and a second member 130 of a slideable handle assembly 104 can be of any length, and a first member 120 of a slideable handle assembly 104 can be have a different length than a second member 130 of a slideable handle assembly 104.

It will be appreciated that one or both of a first member 120 and a second member 130 of a slideable handle assembly 104, as described above, can include two or more slideable segments. For example first member 120 can include segments 120A, 120B that can be configured to slideably fit one within another, and second member 130 can include segments 130A, 130B that can be configured to slideably fit one within another, and in turn the first member segments 120A, 120B can be configured to slideably fit within the second member 130 or to slideably fit within a segment 130A, 130B of the second member 130.

In some embodiments a first segment 120A, 130A, respectively, of a first member 120 or a second member 130 can have a smaller diameter than a second segment 120B 130B, which may allow the first segment 120A, 130A to be slideable within the second segment 120B, 130B of, respectively, the first member 120 and the second member 130. In an example, a first segment 120A of a first member 120 can have a smaller diameter than a second segment 120B of the first member 120, and a first segment 120A of the first member 120 can be slideably received within the second segment 120B of the first member 120. A segment 120A, 120B of the first member 120 can have a smaller diameter than a second member 130 or a segment 130A, 130B of a second member 130, and a first member segment 120A, 120B can be slideably received within the second member 130 or a segment 130A, 130B of the second member 130. Similarly, a first segment 130A of a second member 130 can have a smaller diameter than a second segment 130B of the second member 130 and can be slideably received within the second segment 130B of the second member 130. In an example, a first member segment 120A, 120B having the largest diameter among all of the segments 120A, 120B of the first member 120 can be configured to be slideably received at least partially within a second member 130 or within a second member segment 130A, 130B having the smallest diameter among all segments 130A, 130B of the second member 130.

It will be appreciated that more or fewer segments 120A, 120B of a first member 120 and more or fewer segments 130A, 130B of a second member 130 of a slideable handle assembly 104 can be provided as required or desirable for a particular use. In particular, the number of slideable segments of a first member 120 and a second member 130 can be varied as required or desired to provide the desired “throw”, momentum or force to manipulate an object coupled to the slap hammer tool 100. Also, the length of any slideable segment 120A, 120B, 130A, 130B of a first member 120 and a second member 130 of a telescoping handle assembly 104 can be varied as required or desired to provide the desired “throw”, momentum or force to manipulate an object coupled to the slap hammer tool 100.

In some embodiments the handle assembly 104, including one or both of the first member 120 and the second member 130 of the slideable handle assembly 104, can include a weight or be fabricated from a weight or a heavy material. In some embodiments a first member 120 of the handle assembly can include a stop 126, 128 disposed on or coupled to the first member 120. In some embodiments a first member 120 of a slideable handle assembly 104 can include a first stop 126, disposed proximal to a second member 130 of the handle assembly 100 and a second stop 128 disposed proximal to a connecting portion 102 of the slap hammer tool 100.

Therefore, when a connecting portion 102 of a slap hammer tool 100 is detachably coupled to a first member 120 of a handle assembly 104, the handle assembly 104 can be actuated by a user, such as by slideably moving a second member 130 of the handle assembly 104 over at least a portion of the first member 120 of the handle assembly 100, the second member 130 of the handle assembly 104 can contact a stop 126, 128 disposed on or coupled to the first member 120 of the handle assembly 104. When the second member 130 of the handle assembly 104 contacts a stop 126, 128 disposed on the first member 120, a force f in the direction of the slideable movement of the second member 130 can be applied to the stop 126, 128, the force being transmitted to a first end 108 of the connecting portion 102, to a second end 110 of connecting portion 102, and to an object engaged with or coupled to the second end 110 of connecting portion 102, for example an object coupled to the connecting portion 102 through an engagement or connecting feature 116 of the connecting portion 102.

In some embodiments a handle assembly 104 can be actuated by slideable movement of a second member 130 of a handle assembly 104 in a first direction D1 over a portion of a first member 120 of the handle assembly 104 to apply a force f to a stop 126, 128 disposed on or coupled to the first member 120. In other embodiments a handle assembly can be actuated by slideable movement of a second member 130 of a handle assembly 104 in a second direction D2, opposite the first direction D1, over a portion of a first member 120 of the handle assembly 104 to apply a force f to a stop 126, 128 disposed on or coupled to the first member 120 or to apply a force f to a stop 112, 114 on the connecting portion 102. It will be understood that any embodiment of a handle assembly 104 and a connecting portion 102 can be configured such that slideable movement of the handle assembly 104 in relation to the connecting portion 102, including slideable movement of a second member 130 in relation to one or both of a first member 120 of the handle assembly 104 and a connecting portion 102, can include slideable movement in one or both of the first direction D1 and the second direction D2.

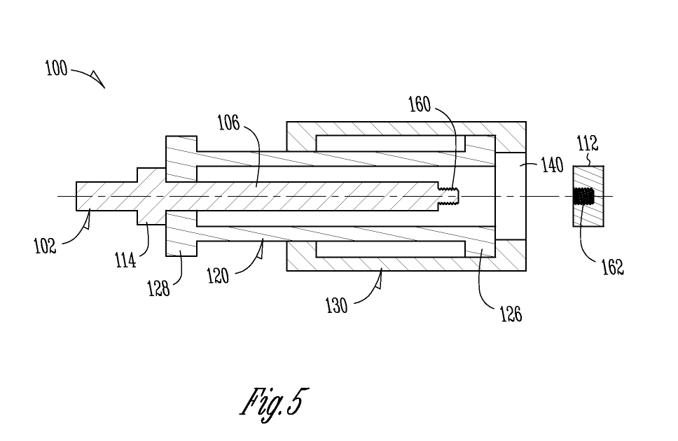

In one or more embodiments of a slap hammer tool 100 a connecting portion 102, as previously described, can be configured to be detachably coupled to a handle assembly 104. An example of a detachable handle assembly coupled to a connecting portion 102 is illustrated in FIG. 5. In some embodiments, a connecting potion 102 can be configured to be detachably coupled to a handle assembly 104 through a threaded connection 160, 162. In some embodiments a connecting portion 102 can be configured to be detachable coupled to a handle assembly 104 through a detachable stop 112, 114. In certain embodiments a connecting portion 102 coupling means 160 can include external threads and the detachable stop 112, 114 coupling means 162 can include internal threads for threadably coupling the stop 112, 114 to the connecting portion 102. In other embodiments a connecting portion 102 coupling means 160 can include internal threads and a detachable stop 112, 114 coupling means 162 can include external threads for threadably coupling the detachable stop 112, 114 to the connecting portion 102. In such configurations, connecting portion 102 detached from the detachable stop 112,114 can be coupled to handle assembly 104 by sliding connecting portion 102 through an opening 122, 132, 140 of handle assembly 104, and then coupling the detachable stop 112, 114 to the connecting portion 102.

In other embodiments, connecting portion 102 can be detachably coupled to handle assembly 104 through a detachable coupling means 160, 162. In some embodiments, connecting portion 102 can include a coupling 160, such as a threaded portion, and handle assembly 104 can include a corresponding coupling 162 configured to mate with connecting portion coupling 160. In some embodiments connecting portion coupling 160 can include one of external threads or internal threads, and the handle assembly coupling 162 can include the other one of external threads or internal threads. It will be understood that any number of other means, fasteners and coupling mechanisms 160, 162 can be used for detachable coupling a handle assembly 104 to a connecting portion 102.

A detachable handle assembly 104 can be configured to include a slideable first member 120 and a slideable second member 130 configured for slideable movement relative to the first member 120. In some embodiments each of the first member 120 and the second member 130 can be configured for slideable movement relative to the connecting portion 102. In some embodiments slideable movement of the slideable first member 120 relative to the connecting portion 102 can be configured to be independent of slideable movement of the second member 130 relative to the connecting portion 102. In some embodiments slideable movement of the slideable second member 130 relative to the connecting portion 102 can be configured to be independent of slideable movement of the first member 120 relative to the connecting portion 102. In some embodiments, one or both of a slideable first member 120 and a slideable second member 130 of the handle assembly 104 can include tubular segments. In some embodiments a first member 120 of the handle assembly 104 can be configured to be at least partially slideably received within a tubular second member 130 of the handle assembly 104. In other embodiments, a handle assembly 104 can include at least two tubular members 120, 130, such that a tubular first member 120 can be configured to be slideably received within the tubular second member 130.

It will be appreciated that one or both of a first member 120 and a second member 130 of a slideable handle assembly 104, as described above, can include two or more slideable segments. For example first member 120 can include segments 120A, 120B that can be configured to slideably fit one within another, and second member 130 can include segments 130A, 130B that can be configured to slideably fit one within another, and in turn the first member segments 120A, 120B can be configured to slideably fit within the second member 130 or to slideably fit within a segment 130A, 130B of the second member 130.

In some embodiments a first segment 120A, 130A, respectively, of a first member 120 or a second member 130 can have a smaller diameter than a second segment 120B 130B, which may allow the first segment 120A, 130A to be slideable within the second segment 120B, 130B of, respectively, the first member 120 and the second member 130. In an example, a first segment 120A of a first member 120 can have a smaller diameter than a second segment 120B of the first member 120, and a first segment 120A of the first member 120 can be slideably received within the second segment 120B of the first member 120. A segment 120A, 120B of the first member 120 can have a smaller diameter than a second member 130 or a segment 130A, 130B of a second member 130 and a first member segment 120A, 120B can be slideably received within the second member 130 or second member segment 130A, 130B. Similarly, a first segment 130A of a second member 130 can have a smaller diameter than a second segment 130B of the second member 130 and can be slideably received within the second segment 130B of the second member 130. In an example, a first member segment 120A, 120B having the largest diameter among all of the segments 120A, 120B of the first member 120 can be configured to be slideably received at least partially within a second member 130 or within a second member segment 130A, 130B having the smallest diameter among all segments 130A, 130B of the second member 130.

It will be appreciated that more or fewer segments 120A, 120B of a first member 120 and more or fewer segments 130A, 130B of a second member 130 of a slideable handle assembly 104 can be provided as required or desirable for a particular use. In particular, the number of slideable segments of a first member 120 and a second member 130 can be varied as required or desired to provide the desired “throw”, momentum or force to manipulate an object coupled to the slap hammer tool 100. Also, the length of any slideable segment 120A, 120B, 130A, 130B of a first member 120 and a second member 130 of a telescoping handle assembly 104 can be varied as required or desired to provide the desired “throw”, momentum or force to manipulate an object coupled to the slap hammer tool 100.

In some embodiments the handle assembly 104, including one or both of the first member 120 and a second member 130 of the handle assembly 104, can include a weight or be fabricated from a weight or a heavy material. In some embodiments a first member 120 of the handle assembly can include a stop 126, 128 disposed on or coupled to the first member 120. In some embodiments a first member 120 of a slideable handle assembly 104 can include a first stop 126, disposed proximal to a second member 130 of the handle assembly 100, and a second stop 128 disposed proximal to the connecting portion 102.

Therefore, when a connecting portion 102 is detachably coupled to a slideable handle assembly 104 the slideable handle assembly 104 can be actuated by a user for slideable movement relative to the connecting portion 102, such as by sliding a first member 120 over a coupling segment of the slideable handle assembly 104 and by sliding, concurrently or consecutively with sliding of the first member 120, a second member 130 of the handle assembly 104 over at least a portion of the first member 120 of the slideable handle assembly 104, the second member 130 of the handle assembly 104 can contact a stop 126, 128 disposed on or coupled to the first member 120 of the slideable handle assembly 104. When the second member 130 of the slideable handle assembly 104 contacts a stop 126, 128, a force f in the direction of the slideable movement of the second member 130 can be applied to the stop 126, 128, the force being transmitted to a first end 108 of connecting portion 102, a second end 110 of connecting portion 102, and an object engaged with the second end 110 of connecting portion 102, for example an object coupled to the connecting portion 102 through an engagement or connecting feature 116 of the slap hammer tool 100.

In some embodiments a handle assembly 104 can be actuated by slideable movement of a first member 120 in a first direction D1 over a portion of a coupling segment of a handle assembly 104 and slideable movement of a second member 130 of a slideable handle assembly 104 in the first direction D1 over a portion of the first member 120 of the slideable handle assembly 104 to apply a force f in the direction D1 to a stop 126, 128 disposed on or coupled to the first member 120. In other embodiments a slideable handle assembly 104 can be configured to be actuated by slideable movement of a first member 120 in a second direction D2, opposite the first direction D1, over a portion of a coupling segment of the slideable handle assembly 104 and by slideable movement of a slideable second member 130 of a handle assembly 104 in the second direction D2 over a portion of the first member 120 of the slideable handle assembly 104 to apply a force f in the direction D2 to a stop 126, 128 disposed on or coupled to the first member 120 or to apply a force f in the direction D2 to a stop 112, 114 on the connecting portion 102. It will be understood that any embodiment of a handle assembly 104 and a connecting portion 102 as described herein can be configured such that slideable movement of the handle assembly 104 in relation to the connecting portion 102, including slideable movement of a first member 120 and a second member 130 in relation to a connecting portion 102, can include slideable movement in one or both of a first direction D1 and a second direction D2.

In one or more embodiments a slap hammer tool 100, as previously described, a connecting portion 102 can be configured for engagement and disengagement with an object, such as a surgical instrument or a device used in connection with a surgical procedure. An engagement feature 116 of the slap hammer tool 100 can be provided by various releasable connecting mechanisms, including, for example, a threaded connection or releasable coupling mechanism (e.g., as described in U.S. Patent App. Pub. No. 20130204265), allowing a releasable connection to be established with a slap hammer tool that is attachable to and removable from an object. Upon releasable coupling of an object to the slap hammer tool, the slap hammer tool 100 can be actuated as described above to provide force to the object in a desired direction. The releasable coupling of the slap hammer tool can be disengaged to detach the slap hammer tool from the object.

In one or more embodiments of a slap hammer tool 100 a connecting portion 102 can include an engagement feature 116 configured for engageable mating with a receiving member or receiving feature of an object, such as a surgical instrument or a surgical device. In some embodiments the removability and attachment of the engagement feature 116 from or to another object, respectively, can be provided by one or more receiving mechanisms in the object, such as threads, turn fasteners, cam fasteners, sliding fasteners, or push-button fasteners configured to receive an engagement feature 116 disposed on or coupled to a connecting end 110 of a connecting portion 102 of a slap hammer tool 100.

In some embodiments a handle assembly can include a drive feature 170 configured to engage an engagement feature 116 of the slap hammer tool 100 with a receiving feature of an object to be coupled to the slap hammer tool 100. A receiving feature of an object to be coupled to the slap hammer tool 100 can be configured (e.g., include internal threads, channels, or mechanisms) to engage an engagement feature 116 of the slap hammer tool 100 when a drive feature 170 of the slap hammer tool 100 is rotated in a first engaging direction (e.g., a clockwise direction). In a similar manner, the receiving feature of the object can be configured to disengage the engagement feature 116 of the slap hammer tool 100 when the drive feature 170 of the slap hammer tool is rotated in a second disengaging direction (e.g., a counter-clockwise direction).

Although the present slap hammer tool has been described with reference to a manipulation of an object used in connection with a surgical procedure or to perform various surgical techniques, it will be understood that the slap hammer tool as disclosed herein can be used with other objects and in connection with other manipulation procedures, methods and techniques in which a slap hammer tool can be or is customarily used.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more” or “a plurality.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described embodiments (or one or more aspects thereof) can be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment.

Thus, the following claims are hereby incorporated into the Detailed Description as examples or embodiments, with each claim standing on its own as a separate example, and it is contemplated that such embodiments can be combined with each other in various combinations or permutations. The scope of the invention should be determined with reference to the appended claims, along with the full scope of equivalents to which such claims are entitled.

What is claimed is:

1. A slap hammer tool system, comprising:
a guide rod; and
a handle assembly configured to be coupled to the guide rod, including a first member and a second member, the first member telescopically movable relative to the second member.

2. The system of claim 1, wherein the handle assembly is configured to be telescopically coupled to the guide rod.

3. The system of claim 1, wherein in a coupled configuration the guide rod is telescopically movable relative to one or both of the first member and the second member.

4. The system of claim 1, wherein in a coupled configuration at least a portion of the guide rod is nested within a chamber of the first member and at least a portion of the first member is nested within a chamber of the second member.

5. The system of claim 1, wherein in a coupled configuration at least a portion of the guide rod is telescopically extended from a chamber of the first member and at least a portion of the first member is telescopically extended from a chamber of the second member.

6. The system of claim 1, wherein in a coupled configuration the first member is configured to impact a striking feature of the guide rod when the first member is telescopically moved relative to the guide rod.

7. The system of claim 1, wherein the second member is configured to impact a striking feature of the first member when the second member is telescopically moved relative to the first member.

8. The system of claim 1, the first member including a first stop and a second stop, and wherein the second member is configured to impact the first stop when the second member is telescopically moved in a first direction relative to the first member, and the second member is configured to impact the second stop when the second member is telescopically moved in a second direction, opposite the first direction, relative to the first member.

9. The system of claim 1, the first member comprising a first segment and a second segment, the first segment configured to be telescopically movable relative to the second segment, the second segment configured to be telescopically movable relative to the second member.

10. A slap hammer tool, comprising:
a guide rod; and
a handle assembly coupled to the guide rod, including a first member and a second member slideably movable relative to each other in response to a user manipulation.

11. The slap hammer tool of claim 10, wherein the first member is at least partially received within a chamber of the second member.

12. The slap hammer tool of claim 10, wherein the guide rod is at least partially received within a chamber of the handle assembly.

13. The slap hammer tool of claim 10, wherein the first member, the second member, or both, is slideably movable relative to the guide rod in response to the user manipulation.

14. The slap hammer tool of claim 10, wherein the handle assembly is slideably moveable relative to the guide rod in a first direction to a first stop coupled to the guide rod, and the handle assembly is slideably moveable relative to the guide rod in a second direction, opposite the first direction, to a second stop coupled to the guide rod.

15. The slap hammer tool of claim 10, the guide rod including a striking feature, the handle assembly further including an impacting feature configured to contact the striking feature when the handle assembly is slideably moved in response to the user manipulation.

16. The slap hammer tool of claim 15, wherein the impacting feature is disposed adjacent an interior chamber of the handle assembly.

17. The slap hammer tool of claim 15, wherein the impacting feature is disposed on an exterior surface of the handle assembly.

18. A method, comprising:
coupling a slap hammer tool to an object, the slap hammer including a guide rod and a telescoping handle assembly coupled to the guide rod, the telescoping handle assembly including a first member and a second member telescopically engaged with each other; and
slideably moving the first member or the second member in a direction to apply a force to the object in the direction of slideable movement of the first member or second member.

19. The method of claim 18, wherein the step of slideably moving includes slideably moving the first member or the second member relative to the guide rod.

20. The method of claim 19, wherein slideably moving the first member or the second member relative to the guide rod includes slideably moving the first member or the second member away from the object or toward the object.
